(19) United States
(12) Patent Application Publication
(10) Pub. No.: US 2023/0270983 A1
(43) Pub. Date: Aug. 31, 2023

(54) WEARABLE PHALLIC LUBRICATION DELIVERY SYSTEM

(71) Applicant: Sameer Mahadeorao Naranje, Vestavia, AL (US)

(72) Inventor: Sameer Mahadeorao Naranje, Vestavia, AL (US)

(21) Appl. No.: 18/174,786

(22) Filed: Feb. 27, 2023

Related U.S. Application Data
(60) Provisional application No. 63/314,847, filed on Feb. 28, 2022.

(51) Int. Cl. A61M 35/00; A61F 6/04

(52) U.S. Cl. CPC A61M 35/10; A61F 6/04; A61M 2210/167

(57) ABSTRACT

A wearable phallic lubrication delivery system providing a sheath or framing extending between a closed distal end and an open proximal end for slidably receiving a phallus. An arcuate, circumferential bladder depends from the open proximal end, proximate thereof. A conduit is fluidly coupled to the bladder and extends longitudinally, along the sheath or framing, from adjacent the open proximal end to adjacent the closed distal end. The conduit is connected to but exclusive the sheath or framing. An outlet is provided at the distal end of the conduit, adjacent the tip of the closed proximal end, so that compression of the bladder urges fluid therein through the conduit and out the outlet, thereby simulating ejaculation.

### CROSS-REFERENCE TO RELATED APPLICATION

This application claims the benefit of priority of U.S. Provisional Application No. 63/314,847, filed 28-FEB-2022, the contents of which are herein incorporated by reference.

### BACKGROUND OF THE INVENTION

The present invention relates to methods and apparatus for providing condom lubrication and, more particularly, to a combination condom and lubrication delivery system and/or wearable phallic lubrication delivery system.

During sexual activities lubrication is needed or preferred but sometimes natural lubrication is not enough, thereby creating a desire for artificial or extrinsic lubrication.

Conventionally, artificial lubrication is typically applied over the sheathed penis by hand, resulting in an interruption of the sexual experience. Also, with condoms, the other partner cannot enjoy the pleasure of ejaculation in their sexual organs.

Current condoms with superficially applied lubrication so do not remain lubricated for a long time, at least for prolonged sex. Secondly, current condoms do not have a mechanism to simulate ejaculation.

Current inventive solutions have either lubrication pouches/reservoirs attached at the tip around glans penis area or in the shaft of condom itself, resulting in thickening of the condom and hence reduced sensations for the person using such condoms. As a result, whatever is gained by adding lubrication through bulk is lost by the sensation reduction.

Also, the amount of lubrication that can be put in these pouches/reservoirs is limited. There is also a need sometimes to puncture these pouches at multiple places for use, resulting in risk of contamination or loss of integrity of the condom.

A need exists for a wearable phallic lubrication delivery system providing semi-continuous and selectively simulated ejaculations.

### SUMMARY OF THE INVENTION

In one aspect of the present invention, a wearable phallic lubrication delivery system includes the following: a covering of a phallus, the covering extending between a distal end and an open proximal end; a bladder disposed proximal of and circumscribing the open proximal end; a conduit fluidly coupled to and extending perpendicular the bladder along a longitudinal surface of the covering; and an outlet of the conduit disposed adjacent the distal, whereby pressure along a surface of the bladder proximal the open proximal end selectively simulates ejaculation of the phallus.

In another aspect of the present invention, the wearable phallic lubrication delivery system further includes wherein the covering is a condom and the distal end is closed, wherein a conduit sidewall of the conduit is exclusive a sheathing sidewall of the condom; and an occluder removable plugging the outlet.

In yet another aspect of the present invention, the wearable phallic lubrication delivery system further includes, wherein the covering comprises a plurality of longitudinal and arcuate framing members, wherein the conduit is attached along one of the longitudinal framing members, wherein one of the arcuate framing members defines an open distal end; further including a pliable mesh tip covering the open distal end; and an occluder removable plugging the outlet.

These and other features, aspects and advantages of the present invention will become better understood with reference to the following drawings, description, and claims.

### BRIEF DESCRIPTION OF THE DRAWINGS

FIG. 1 is a perspective view of an exemplary embodiment of the present invention.

FIG. 2 is a side elevation view of an exemplary embodiment of the present invention.

FIG. 3 is a front elevation view of an exemplary embodiment of the present invention.

FIG. 4 is a section view of an exemplary embodiment of the present invention, taken along line 4-4 in FIG. 3.

FIG. 5 is a detailed section view of an exemplary embodiment of the present invention.

FIG. 6 is a perspective view of an exemplary embodiment of the present invention.

FIG. 7 is a side elevation view of an exemplary embodiment of the present invention.

FIG. 8 is a side elevation view of an exemplary embodiment of the present invention, shown in use with a mesh tip 26 stretched.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a wearable phallic lubrication delivery system providing a sheath or framing extending between a closed distal end and an open proximal end for slidably receiving a phallus. An arcuate, circumferential bladder depends from the open proximal end, proximate thereof. A conduit is fluidly coupled to the bladder and extends longitudinally, along the sheath or framing, from adjacent the open proximal end to adjacent the closed distal end. The conduit is connected to but exclusive the sheath or framing. An outlet is provided at the distal end of the conduit, adjacent the tip of the closed proximal end, so that compression of the bladder urges fluid therein through the conduit and out the outlet, thereby simulating ejaculation.

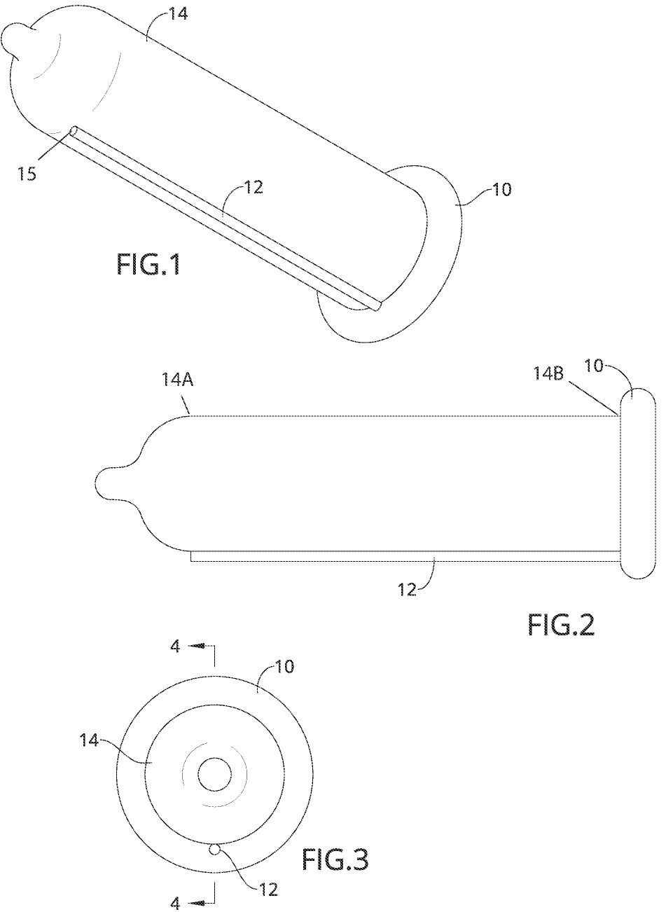

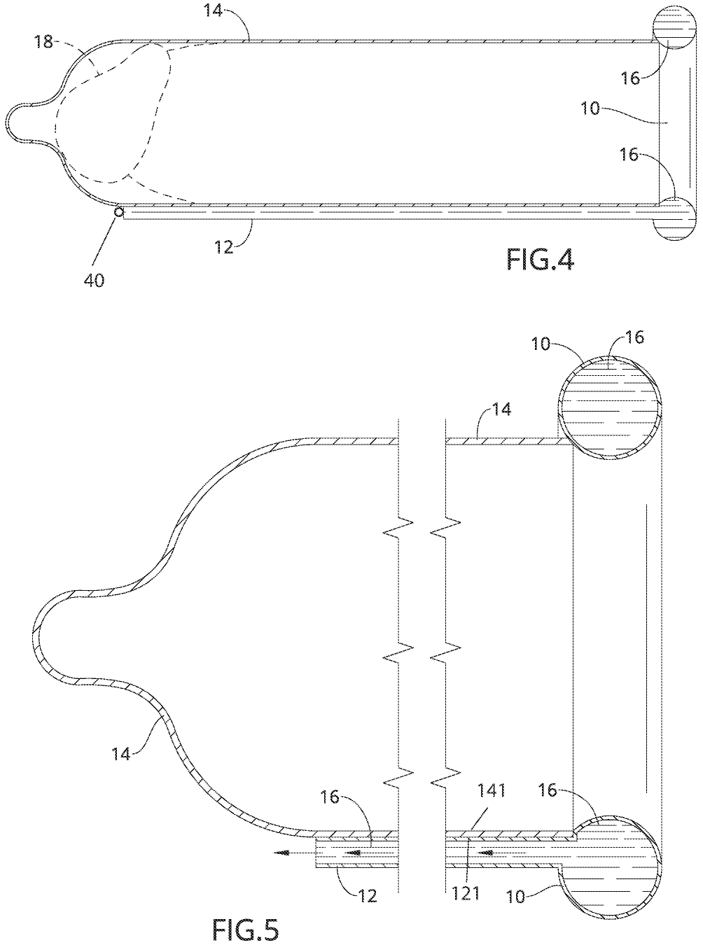

Referring to FIGS. 1 through 5, the present invention may include a nonporous sheath member 14 (or condom) shaped to cover the glans and body portion of a penis 18, wherein the condom 14 extends between an open end 14A and a closed end 14B. A conduit 12 may extend along a barrel of the sheath member 14, for example, generally longitudinally from the open end 14A toward the closed end 14B. Adjacent to the open end 14A, an arcuate bladder 10 circumferentially engages the open end 14A so as to depend from the open end 14A - i.e., is proximate the open end 14A.

The conduit 12 may be fluidly coupled to the bladder 10, thereby allowing for a flow of fluid 16 (including but not limited to lubricant) along the barrel of the condom 14 from the bladder 10 to the distal end of the condom 14. Such flow of the fluid 16 is directional, i.e., one way, such as flowing from the base of the condom 14 toward the tip of the condom 14 during use of the condom 14. An outlet 15 (such as hole) at or near a distal end of the conduit 12 allows the fluid 16 to exit the conduit 12. The conduit 12 may defined by conduit sidewalls 121 that are exclusive of the member sidewalls 141 that define the condom’s barrel. The bladder 10 could be dimensioned to hold three to six milliliters of fluid 16. The conduit 12 extends normal to a plane defined by the arcuate bladder 10.

The disposition of the bladder 14 proximate of the barrel of the condom facilitates a pressure-based release of the fluid 16 during sex; specifically, with each thrust that engenders significant resistance by the orifice of the recipient, the wearer’s body urges against the proximate surface of the bladder 14, thereby causing some of the fluid 16 in the bladder 10 to be urged through the conduit 12 and out of its distal outlet 15. As a result, the wearer can selectively choose when to urge the fluid 16 out of the outlet 15, which would simulate ejaculation. Manual compression by hand on the lube pouch/reservoir/bladder 10 during sex can also release the fluid 16.

The lubrication delivery conduit 12 will have no communication with the condom 14 and will release lubrication 16 outside the condom 14. The lubrication bladder 10 is compressible and with sexual thrusts it will help deliver lubrication 16 the wearer’s partner’s sexual organs, lubricating the sexual organs while selectively simulating one of multiple ejaculations, resulting increased sexual satisfaction and allowing the wearer and partner to experience multiple “orgasms,” which may be otherwise elusive or impossible for the wearer.

Though not shown in FIGS. 1 through 5, the conduit 12 may have its outlet 15 adjacent to the tip of the condom 14 /penis 18, thereby furthering the ejaculation simulation.

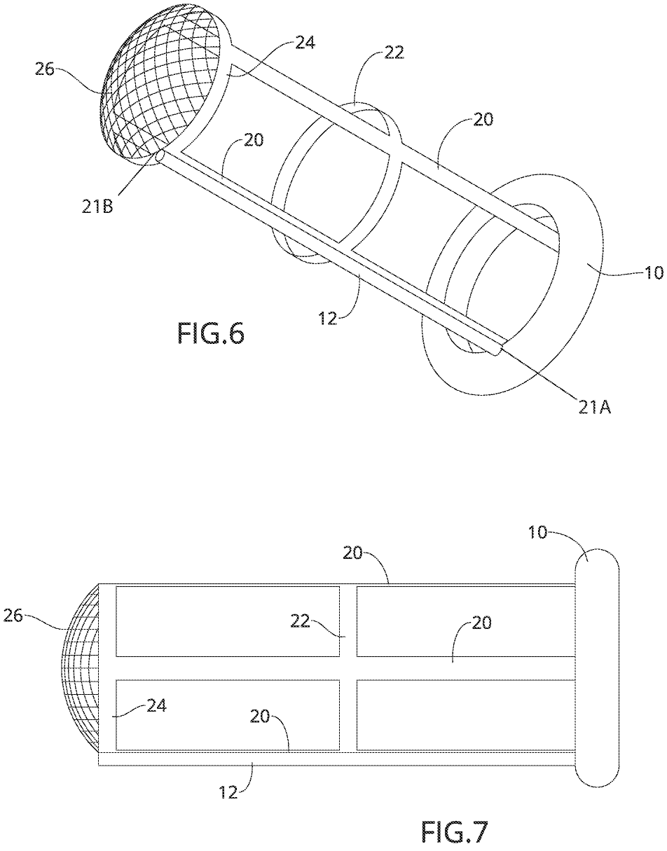

Referring to FIGS. 6 through 8, the present invention may include phallic framing 20 and 22 defining a generally cylindrical space between an open proximal end 21A and a closed distal end 21B, wherein the cylindrical space is dimensioned and adapted to slidably receive, through the open proximal end 21A, a glans and body portion of a penis 18.

The phallic framing 20 and 22 provides structural support for the arcuate bladder 10 and the conduit 12 extending normally therefrom, so they maintain their relative spacing and orientation throughout use, wherein the bladder 10 is proximate the open proximal end 21A of the phallic framing 20 and 22. The phallic framing 20 and 22 may include two or more longitudinal members 20 circumferentially spaced apart along a middle ring member 22. The closed distal end 21B may be defined, at least in part, by a distal ring member 24. A pliable mesh top 26 may cover the opening of the distal ring member 24. The conduit 12 may be rigidly connected to one of the longitudinal members 20. In this embodiment, if a user were interested in enjoying the benefits of the present invention while allowing for their natural/intrinsic ejaculate to enter their partner.

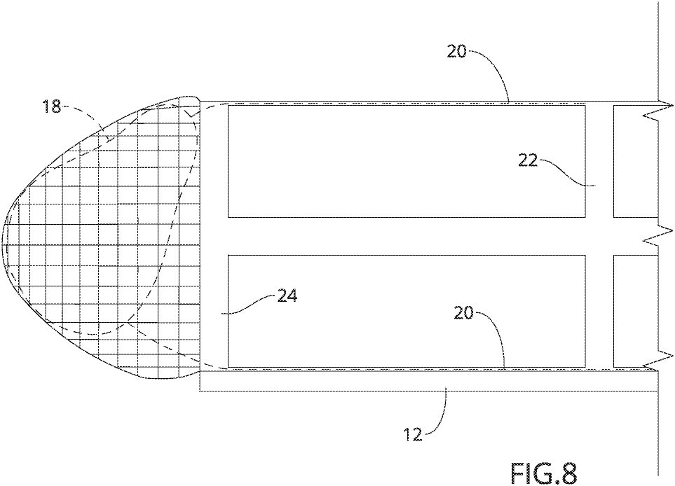

The members 20, 22, and 24 may be soft strapping so that the phallic framing can be comfortably worn on the penis 18, as illustrated in FIG. 8. The pliable mesh tip 26 may be stretchable to accommodate the glans of the penis 18. The strapping/framing 20, 22, and 24 may be made from medical-grade, inert material that is comfortable for the intended use, including but not limited to latex.

A temporary tip occluder 40 or detachable cover can be placed at the tip of the delivery conduit 12 to avoid a fluid 16 leak before use.

Prior use, a wearer may remove the occluder 40. After each use, the present invention is disposed of. During sex, with a thrust that is significantly resisted by the penetrated orifice, the wear selectively urges fluid/lubricant 16 from the bladder/pouch/reservoir 10 caused by the resulting compression against the bladder 10 by a portion of the wearer’s body. The resulting release via the conduit 12 and its outlet 15 into their partner’s sexual organs simulates ejaculation. Manual compression of the lube pouch/reservoir/bladder 10 during sex can also release the lubrication 16.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the present invention.

What is claimed is:

1. A wearable phallic lubrication delivery system comprising:
a covering of a phallus, the covering extending between a distal end and an open proximal end;
a bladder disposed proximal of and circumscribing the open proximal end;
a conduit fluidly coupled to and extending perpendicular the bladder along a longitudinal surface of the covering; and
an outlet of the conduit disposed adjacent the distal,
whereby pressure along a surface of the bladder proximal the open proximal end selectively simulates ejaculation of the phallus.

2. The wearable phallic lubrication delivery system of claim 1, wherein the covering is a condom and the distal end is closed.

3. The wearable phallic lubrication delivery system of claim 2, wherein a conduit sidewall of the conduit is exclusive a sheathing sidewall of the condom.

4. The wearable phallic lubrication delivery system of claim 3, further comprising an occluder removable plugging the outlet.

5. The wearable phallic lubrication delivery system of claim 1, wherein the covering comprises a plurality of longitudinal and arcuate framing members.

6. The wearable phallic lubrication delivery system of claim 5, wherein the conduit is attached along one of the longitudinal framing members.

7. The wearable phallic lubrication delivery system of claim 6, wherein one of the arcuate framing members defines an open distal end.

8. The wearable phallic lubrication delivery system of claim 7, further comprising a pliable mesh tip covering the open distal end.

9. The wearable phallic lubrication delivery system of claim 8, further comprising an occluder removable plugging the outlet.
